(19) United States
(12) Patent Application Publication
(10) Pub. No.: US 2021/0100586 A1
(43) Pub. Date: Apr. 8, 2021

(54) Method for percutaneous fixation of a humeral head fracture and related device

(71) Applicant: ORTHOFIX S.R.L., Bussolengo (IT)

(72) Inventor: Daniele Venturini, Povegliano Veronese (VR) (IT)

(21) Appl. No.: 16/593,091

(22) Filed: Oct. 4, 2019

(51) Int. Cl. A61B 17/64; A61B 17/88; A61B 17/66

(52) U.S. Cl. CPC A61B 17/6458; A61B 17/66; A61B 17/8861

(57) ABSTRACT

A method for percutaneous fixation of a humeral head fracture and a related external fixation device, the method comprising the steps of:
inserting two oblique wires through the cortical bone of the humerus shaft, said oblique wires being substantially oblique with respect to the humeral axis and crossing the surgical neck toward the humeral head;
inserting two proximal transverse wires through the cortical bone of the humerus, said proximal transverse wires being substantially perpendicular with respect to the humeral axis and being inserted into the humeral head;
inserting two distal transverse wires through the cortical bone of the humerus, said distal transverse wires being substantially perpendicular with respect to the humeral axis and being inserted into the humeral shaft;
connecting the oblique wires, the proximal transverse wires and the distal transverse wires to a common external fixator frame.

### FIELD OF THE INVENTION

The present invention pertains to the field of orthopaedics and relates to a surgical method and device for percutaneous fixation of a humeral head fracture.

### STATE OF THE ART

Proximal humerus fractures are fairly common, especially among older adults, and their treatment sometimes requires a surgical approach, especially to ensure a proper fixation of the fractured bone.

Internal fixation employing locking plates—oftentimes combined with open reduction of the fracture—is today the preferred surgical approach, in particular for complex fractures like for instance three-part and four-part fractures. However, this is an invasive approach and major surgical complications often occur.

Even though external fixation is less invasive, as it preserves vascular supply, maintains fracture hematoma and minimizes the blood loss, the existing devices and techniques do not ensure sufficient stability, at least for complex fractures.

Moreover, the existing devices and techniques are scarcely standardized. The surgeon must adapt the biomechanical construct of the external fixator to the anatomy of the patient and to the actual pattern of the fracture.

Therefore, a need exists for a surgical method of percutaneous fixation of humeral head fractures that is both minimally invasive and stable, and for a corresponding external fixation device that can be applied in a simple, standardized and reproducible manner at least to the most common humeral fracture patterns.

### SUMMARY OF THE INVENTION

The afore-mentioned need is met by a method for percutaneous fixation of a humeral head fracture, said method comprising the steps of:
inserting two oblique wires through the cortical bone of the humerus, said first oblique wires entering the bone at a site distal from the surgical neck and targeting the humeral head;
inserting two proximal transverse wires through the cortical bone of the humerus, said proximal transverse wires entering the bone at a site proximal to the surgical neck and targeting the humeral head;
inserting two distal transverse wires through the cortical bone of the humerus, said distal transverse wires entering the bone at a site distal to the surgical neck and targeting the humeral shaft;
connecting the oblique wires, the proximal transverse wires and the distal transverse wires to a common external fixator frame.

In the step of inserting the oblique wires, each of the oblique wires may be inserted until its tip is in the subchondral area of the humeral head.

In the step of inserting the oblique wires, each wire may be inserted from anterior to posterior.

In the step of inserting the oblique wires, each wire may be inserted with an inclination comprised between 15° and 25° with respect to the frontal plane.

In the step of inserting the oblique wires, the two wires may be inserted substantially parallel to each other; in the step of inserting the proximal transverse wires, the two wires may be inserted substantially parallel to each other; in the step of inserting the distal transverse wires, the two wires may be inserted substantially parallel to each other.

The method may further comprise a preliminary step of performing a closed reduction of the fracture. Obviously, said step must be performed before the steps of fixing the humeral head fracture.

In this case, the distal transverse wires may be inserted in the skin proximal to the oblique wires, whereas they may enter the bone distally to the oblique wires.

Alternatively, the method may comprise a step of performing an open reduction of the fracture.

The step of performing the open reduction of the fracture may comprise making a deltopectoral incision, wherein the deltopectoral incision may be kept open during at least one of the steps of inserting the oblique wires, inserting the distal transverse wires and inserting the proximal transverse wires.

The steps of connecting the oblique wires, the proximal transverse wires and the distal transverse wires to a common external fixator frame may comprise a sub-step of connecting the proximal transverse wires and the distal transverse wires at the two opposite ends of a main rod.

The steps of connecting the oblique wires, the proximal transverse wires and the distal transverse wires to a common external fixator frame may comprise a sub-step of connecting the oblique transverse wires to the main rod by means of an auxiliary rod, the auxiliary rod departing anteriorly with respect to the main rod.

The steps of connecting the oblique wires, the proximal transverse wires and the distal transverse wires to a common external fixator frame may comprise a sub-step of connecting each pair of oblique, proximal transverse or distal transverse wires to a corresponding wire locking clamp.

The steps of connecting the oblique wires, the proximal transverse wires and the distal transverse wires to a common external fixator frame may comprise a further step of connecting each wire locking clamp to a main or auxiliary rod by means of a rod locking clamp.

Preferably, each of the oblique wires, the proximal transverse wires and the distal transverse wire is a bicortical wire.

Preferably, each of the oblique wires, the proximal transverse wires and the distal transverse wire is partially threaded.

The above-mentioned need is also met by a method for percutaneous fixation of a humeral head fracture, said method comprising the following steps to be carried out after the reduction of the fracture:
inserting two oblique wires through the cortical bone of the humerus shaft, said oblique wires being substantially oblique with respect to the humeral axis and crossing the surgical neck toward the humeral head;
inserting two proximal transverse wires through the cortical bone of the humerus, said proximal transverse wires being substantially perpendicular with respect to the humeral axis and being inserted into the humeral head;
inserting two distal transverse wires through the cortical bone of the humerus, said distal transverse wires being substantially perpendicular with respect to the humeral axis and being inserted into the humeral shaft;
connecting the oblique wires, the proximal transverse wires and the distal transverse wires to a common external fixator frame.

The above-mentioned need is also advantageously met by an external fixation device comprising: two oblique wires arranged to be inserted into the humeral shaft, in a direction substantially oblique with respect to the humeral axis and crossing the surgical neck toward the humeral head; two proximal transverse wires arranged to be inserted into the humeral head in a direction substantially perpendicular with respect to the humeral axis; two distal transverse wires arranged to be inserted into the humeral shaft in a direction substantially perpendicular with respect to the humeral axis; and an external fixator frame stably connecting the oblique wires, the proximal transverse wires and the distal transverse wires.

The external fixator frame may comprise wire locking clamps for holding together each of the pair of oblique, proximal transverse and distal transverse wires.

The external fixator frame may further comprise a main and an auxiliary rod and rod locking clamps to connect the main and auxiliary rods together and each of the wire locking clamps to one of the main or auxiliary rods.

The main rod may be connected to the wire locking clamps relative to the proximal transverse and distal transverse wires, while the auxiliary rod may be connected to the wire locking clamp relative to the oblique wires.

Further characteristics and advantages shall be clearer from the detailed description, outlined hereinafter, of a preferred but not exclusive embodiment of the present finding, with reference to the attached figures provided for exemplifying and non-limiting purposes.

### BRIEF DESCRIPTION OF THE DRAWINGS

In the drawings:

FIG. 1 shows a perspective view of an external fixation device according to the invention applied to a three-part humeral head fracture;

FIG. 2 shows a lateral view of a rod locking clamp of an external fixation device according to the invention;

FIG. 3 shows a lateral view of a wire locking clamp of an external fixation device according to the invention;

FIG. 4 shows a lateral view of a threaded wire of an external fixation device according to the invention;

FIG. 5 shows a perspective view of a wire guide that can be employed in a method according to the invention;

FIG. 6 shows a lateral view of a wire targeting device that can be employed in a method according to the invention, the wire targeting device being in a first configuration;

FIG. 7 shows a lateral view of the wire targeting device of FIG. 6 in a second configuration;

FIG. 8 shows a top view of the wire targeting device of FIG. 6 in its first configuration combined with two wire guides as in FIG. 5;

FIG. 9 illustrates the direction for insertion of a first oblique wire in a method step according to the invention;

FIG. 10 illustrates a deltopectoral access to the bone site for insertion of a first oblique wire in a method step according to the invention;

FIG. 11 shows a step of inserting a first oblique wire according to a method of the invention;

FIG. 12 shows a step of inserting a second oblique wire according to a first variant of a method of the invention;

FIG. 13 shows a step of inserting a second oblique wire according to a first variant of a method of the invention;

FIG. 14 shows the resulting layout after insertion of the second oblique wire according to the method step depicted in FIG. 12 or in FIG. 13, and further shows the position of the wires' tips within the humeral head;

FIG. 15 shows a step of inserting proximal transverse wires according to a method of the invention, with the final position of the wires' tips;

FIG. 16 shows a step of inserting distal transverse wires according to a method of the invention;

FIG. 17 shows a step of inserting the wire pairs in wire locking clamps according to a method of the invention, along with the possibility of bending the wires in order to connect them to other clamps;

FIG. 18 shows a step of tightening the wire locking clamps on wire pairs according to a method of the invention;

FIG. 19 shows a step of connecting the wire locking clamps on corresponding rod locking clamps according to a method of the invention;

FIG. 20 shows a step of connecting the rod locking clamps to a main rod according to a method of the invention;

FIG. 21 shows a step of connecting the rod locking clamps to an auxiliary rod according to a method of the invention.

### DETAILED DESCRIPTION

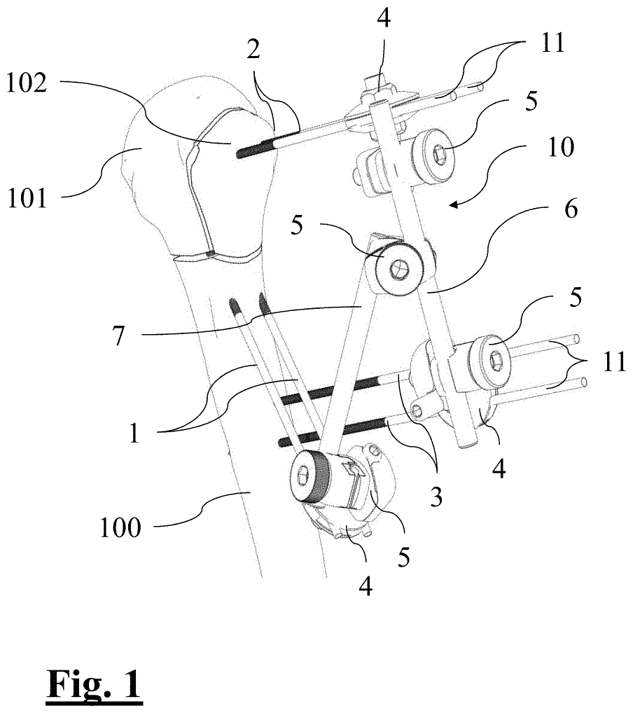

Referring to FIG. 1, an external fixation device 10 according to the invention is shown in its implanted position over a patient's bony structure 100, 101, 102. The bony structure is represented by a humerus 100 which has suffered a three-part fracture at the humeral head 101.

The external fixation device comprises percutaneous wires or pins 1, 2, 3 connected to a common external fixator frame 4, 5, 6, 7.

The device comprises three pair of wires: a pair of oblique wires 1, which enters the bone obliquely distally from the surgical neck and target the humeral head 101; a pair of proximal transverse wires 2, which enters the bone transversally at a third bone fragment 102 and attaches it to the humeral head 101; and a pair of distal transverse wires 3, which enters the bone transversally at the humeral shaft.

The location and orientation of the wires 1, 2, 3 will be further detailed in the following description of the surgical method for implanting the external fixation device 10.

Each of the wire pairs 1, 2, 3 is locked by a single wire locking clamp 4, which in turn is attached to a rod 6, 7 via a rod locking clamp 5. The proximal transverse wires 2 and the distal transverse wires 3 are disposed at the two opposite ends of a main rod 6. The oblique wires 1 are locked to a free end of an auxiliary rod 7 which is attached main rod 6 via a further rod locking clamp 5; in the final configuration of the device, the main rod 6 and the auxiliary rod 7 are set at an angle.

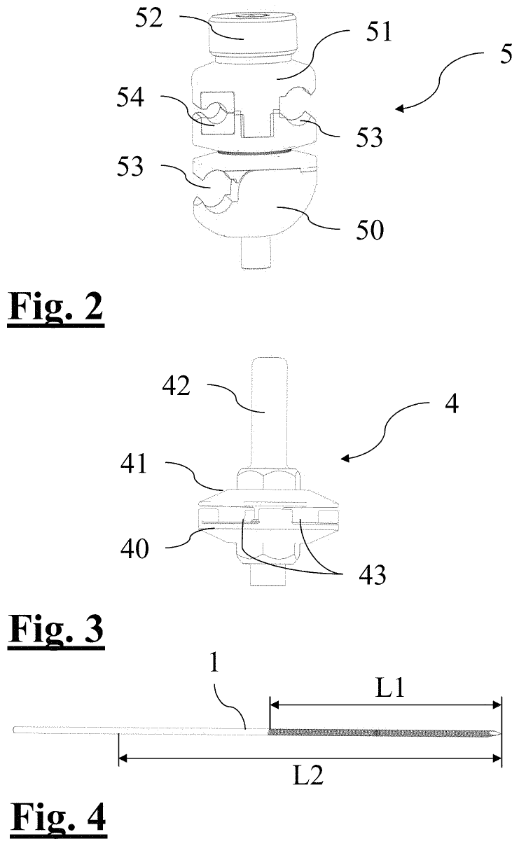

FIG. 2 shows a detailed view of a single rod locking clamp 5. The rod locking clamp comprises two attachments 50, 51, connected by a traversing locking screw with an outstanding tightening knob 52.

The two attachments 50, 51 have jaws defining lateral rod locking seats 53. The lateral rod locking seats 53 are arranged to house either a rod 6, 7 or a stem 42 of wire locking clamp. In a preferred embodiment, both the rods and the stem may have a diameter of 4-8 mm, preferably 6 mm.

The second attachment 51 may have opposite jaws defining a screw locking seat 54; however, said screw locking seat 54 is not employed in the preferred embodiment of the surgical method described in the following.

The two attachments 50, 51 are free to rotate one with respect to the other about the axis of the locking screw in a loose configuration; tightening of the screw locks the respective orientation of the two attachments 50, 51.

Advantageously, the tightening knob 52 allows a manual preliminary closure of the locking screw; the tightening is eventually finalized by means of a wrench that engages with a polygonal socket on top the tightening knob 52.

FIG. 3 shows a detailed view of a single wire locking clamp 4.

The wire locking clamp 4 comprises a lower disk 40 and an upper disk 41 which are coupled to define paired seats 43 for a pair of percutaneous wires 1, 2, 3. The wire locking clamp 4 further comprises an elongated stem 42 which can be locked in a rod locking seat 53 of the rod locking clamp 5.

FIG. 4 shows a detailed view of one of the wires 1, 2, 3 preferably employed in the invention.

The wire or pin 1 is self-drilling and self-tapping. Its threaded length L1 is preferably between 50 mm and 90 mm, most preferably 70 mm. Marks, for instance laser marks, can be provided in order to verify the correct insertion depth. In a preferred embodiment, the wire has two marks: a first mark at a distance L2 of 120 mm from the tip, a second mark (not shown in the drawing) at a distance of 165 mm from the tip.

The total length of the wire is preferably between 250 mm and 350 mm, most preferably 300 mm. The wire diameter is preferably 2.5 mm.

The wires 1, 2, 3 employed in the present invention are preferably bicortical.

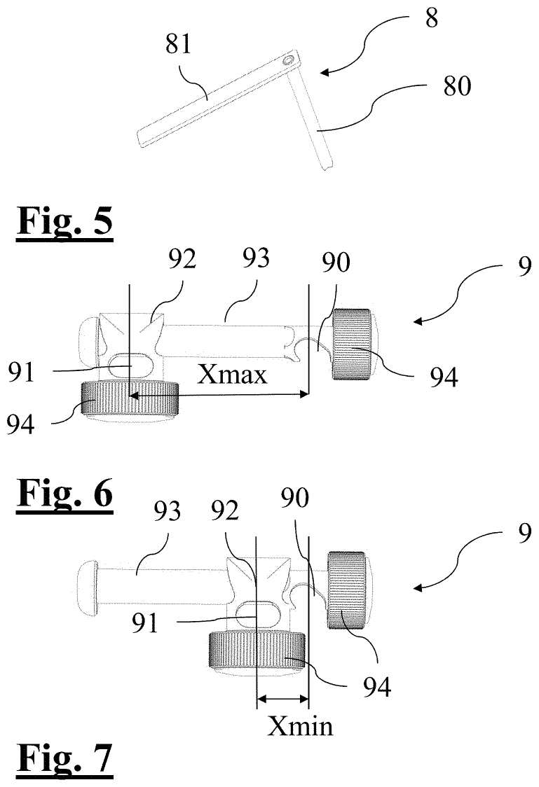

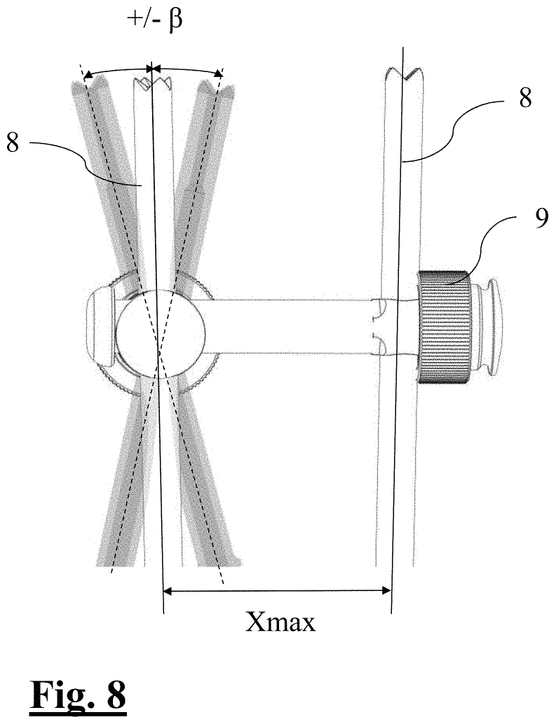

FIG. 5 shows a wire guide 8 that can be employed in the method of the invention.

The wire guide 8 features a sleeve 80 designed to slidingly house a wire 1, 2, 3 during insertion and a reference feature 81 perpendicular to the sleeve 80.

FIGS. 6, 7 show a wire targeting device 9 that can be adopted in the method of the invention. The wire targeting device is meant to support two wire guides 8 in a desired spatial relationship, in order to define the distance and the relative inclination between two of the paired wires (i.e. the oblique wires 1, the proximal transverse wires 2 or the distal transverse wires 3).

The wire targeting device 9 has both a fixed seat 90 and a movable seat 91, each of them being designed to house a corresponding sleeve 80 of a wire guide 8. A locking knob 94 is provided on each of the seats to block the housed sleeve in the desired direction.

The movable seat 91 is provided on a sliding block 92 that can be axially displaced along a guiding rod 93, in order to set the desired distance between the two wires to be inserted. The distance can be varied between a minimum distance Xmin (in the preferred embodiment: 15 mm) and a maximum distance Xmax (in the preferred embodiment: 45 mm).

The fixed seat 90 constrains the sleeve 80 of the wire guide 8 in a preset direction, substantially perpendicular to the guiding rod 93. Conversely, the movable seat 91 is an elongated slot that allows for a slight inclination of the sleeve with respect to the axis perpendicular to the guiding rod 93. Therefore, the two wire sleeves 80 can be set to guide the wires 1, 2, 3 either in a parallel, diverging or converging relationship, according to the type of fracture to be treated.

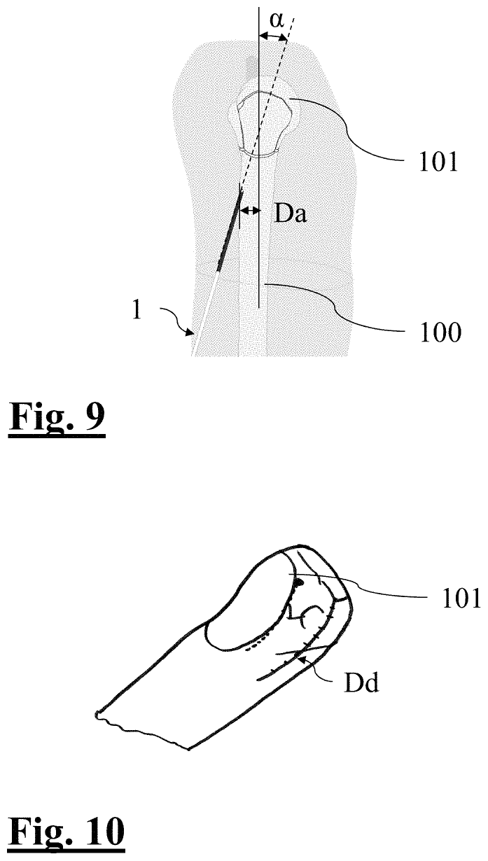

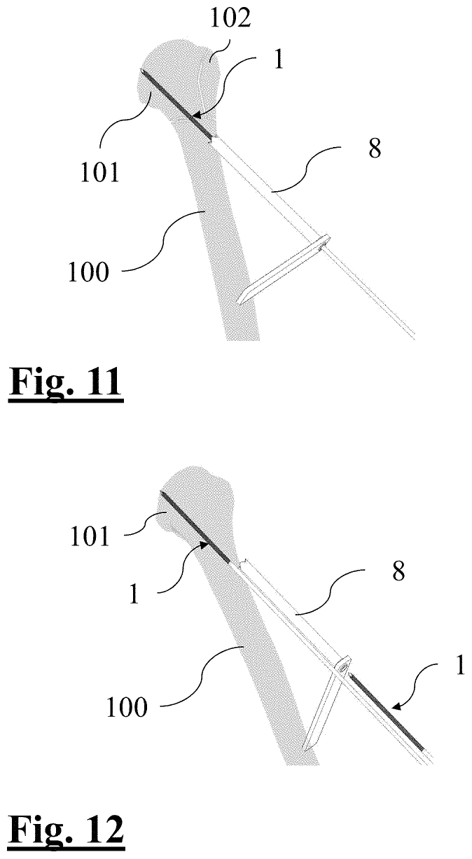

FIGS. 9-21 illustrate a method for fixing a humeral head fracture by implanting the above-mentioned external fixation device 10.

The illustrated method is directed to the fixation of a three-part fracture; however, the same method can be employed for different types of fractures, like for instance two-part fractures or four-part fractures.

The method preferably comprises a preoperative planning, based on images of the fractured bone site of the patient. Anterior-posterior and trans-thoracic or outlet X-ray views can be employed. A CT scan can also be performed, especially in the case of three-part or four-part fractures of the humeral head.

During operation, the patient is preferably placed in a beach chair position; an image intensifier can be positioned on the contralateral side with the X-ray beam positioned over the glenoid to obtain a true AP view. Preferably, the area of the acromioclavicular joint and coracoid should be clearly visible, in order to facilitate percutaneous insertion of the wires.

The method may comprise a preliminary step of reducing the humeral head fracture. The step can be either a closed reduction or an open reduction, depending on the type of fracture, surgeon experience and preference.

If closed reduction is chosen, the reduction is obtained through external manoeuvres meant to relocate the bone fragments in their anatomical position.

Usual fractures of the humeral head have a varus deformity with antero-medial displacement of the humeral shaft. In such cases, reduction can be achieved by combining two manoeuvres. A first manoeuvre reduces the varus deformity by stabilizing the patient's scapula with one hand and moving the arm into abduction. A second manoeuvre reduces the antero-medial displacement of the shaft and the internal rotation of the head by supporting the patient's forearm with one hand and pressing the humeral shaft posertiorly with the other. While performing this second manoeuvre, the arm is kept abducted about 45° and in neutral rotation with gentle pressure.

Alternative manoeuvres can be performed depdening of the pattern of the fracture.

If the reduction is not satisfactory or cannot be obtained with external manipulation, it can be improved using a percutaneous instrument, such as a small hook or a periosteal elevator, inserted through a small deltoid split to reduce the greater tuberosity.

Alternatively, open reduction can be chosen for a wide range of fracture patterns. In this case, the reduction does not differ from other techniques that employ internal fixation.

The fracture is then preferably exposed using a deltopectoral approach. The tuberosities are identified and the rotator interval is opened. Thereafter, the humeral head is reduced by elevation and rotation with one finger or with a blunt instrument.

Possibly after insertion of the oblique wires 1 (described in the following), a hole can be made in the proximal shaft to accommodate two not-absorbable sutures. The tuberosities may be reduced and held in place using the not-absorbable sutures.

The method according to the invention comprises steps of fixing the humeral head fracture by the insertion of the wires 1, 2, 3 and their connection to the external fixator frame 4, 5, 6, 7.

In case of closed reduction, the fixation of the humeral head fracture is performed after the step of reducing the fracture; in case of open reduction, as discussed above, at least the step of reducing the tuberosities by not-absorbable sutures can be performed after a first step of fracture fixation, namely that of inserting the oblique wires 1.

A first fixation step, illustrated in FIGS. 9-14, is the step of inserting the two oblique wires 1, to provide initial fixation of the humeral head 101 to the shaft of the humerus 100.

During this step, it is advantageous to keep the injured arm parallel to the ground, with approximately 45° of abduction, to maintain the reduction.

As depicted in FIG. 9, the oblique wires 1 should be inserted from anterior to posterior with an inclination of an angle α to the humeral diaphysis in order to target the humeral head. The angle α is preferably comprised between 15° and 25°, most preferably 20°.

As depicted in FIGS. 9 and 10, the first of the two oblique wires 1 should enter the bone at a distance Dd distal from the lateral border of the acromion, and at a distance Da anterior to this line. The distance Dd is ideally between 8.5 cm and 9.5 cm, most preferably 9 cm, the distance Da is between 0.5 and 1.5 cm, most preferably 1 cm. The resulting insertion point is usually anterior, in line with the area of deltopectoral approach, thus avoiding nerve injury. The wire is inserted in the direction of the coracoid with a posterior inclination a to allow for anatomical retroversion of the humeral head.

The oblique wires 1 are bicortical, and in their final position their tip should be in the subchondral area of the humeral head, as may be seen in FIG. 14. The wire position can be advantageously checked with the image intensifier and, if necessary, the wire can be repositioned.

The second of the oblique wires 1 should be inserted at close distance from the first, for instance at 1 cm, and is preferably substantially parallel to it.

In case an open reduction is preferred, the oblique wires are then preferably inserted at least 2 cm distal to the deltopectoral incision.

Once the oblique wires 1 have been inserted, the surgeon can check if the fracture is well reduced and that the wires have been correctly inserted by moving the patient's arm in internal and external rotation.

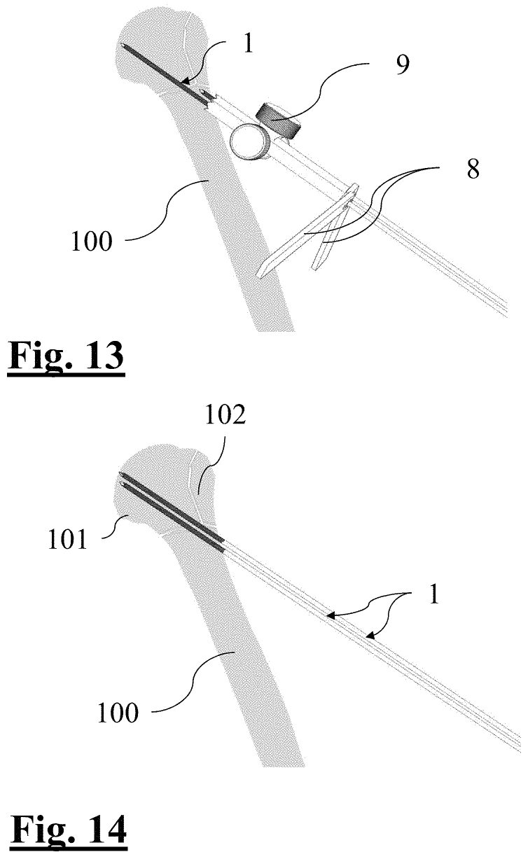

A wire guide 8 may be advantageously employed to guide insertion of the first oblique wire 1. As seen in FIG. 12, a second independent wire guide 8 can be employed to guide insertion of the second oblique wire 1. Alternatively, as seen in FIG. 13, the two wire guides 8 can be connected to a wire targeting device 9 to ensure the desired spatial relationship between the oblique wires 1.

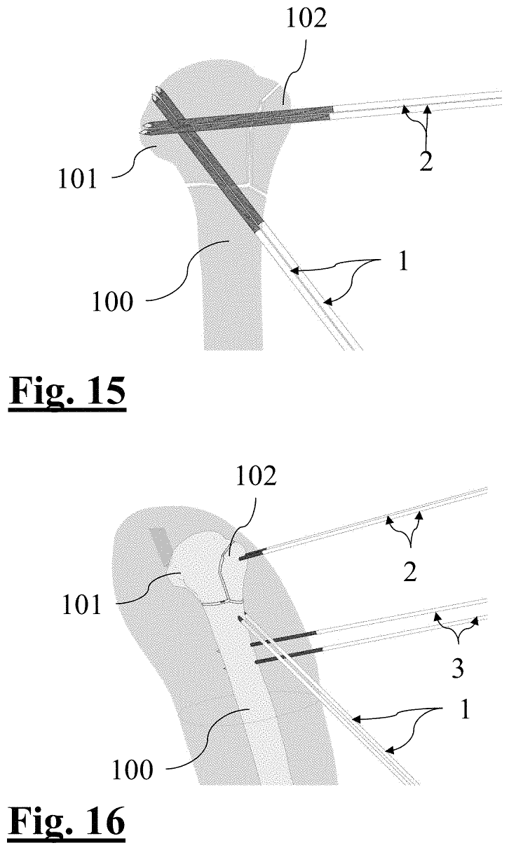

A second fixation step, illustrated in FIG. 15, is the step of inserting the two proximal transverse wires 2 through the greater tuberosity, targeting the subchondral bone of the humeral head 101. The proximal transverse wires 2 have a bicortical figure.

Said second fixation step is preferably performed while keeping the patient's arm 40° abducted.

The proximal transverse wires 2 are inserted from a bone site which is distally closer to the acromial border with respect to the insertion site of the oblique wires. The site is preferably 1 cm to 2 cm distal from the acromial border. The two proximal transverse wires 2 are preferably parallel and they enter the bone at the same distance from the acromial border, being offset from one another of about 1 cm along the anteroposterior direction.

As may be seen in FIG. 15, the proximal transverse wires 2 possibly cross the oblique wires 1 at the humeral head.

A third fixation step, illustrated in FIG. 16, is the step of inserting the two distal transverse wires 3 through lateral cortical bone of the diaphysis, targeting the medial cortical bone. The distal transverse wires 3 have a bicortical figure.

The two distal transverse wires 2 are preferably parallel and they are offset from one another of about 1 cm along the craniocaudal direction.

Wherein a close reduction is performed, the distal transverse wires 3 will be inserted in the skin proximally to the oblique wires 1, whereas they will enter the bone distally to the oblique wires.

Wherein an open reduction is performed, the two distal transverse wires 3 are introduced into the shaft in the proximity of the two oblique wires 1, in a slightly distal site.

After the six wires have been introduced into the fractured bone, the proposed method comprises a step connecting them to a common external fixator frame.

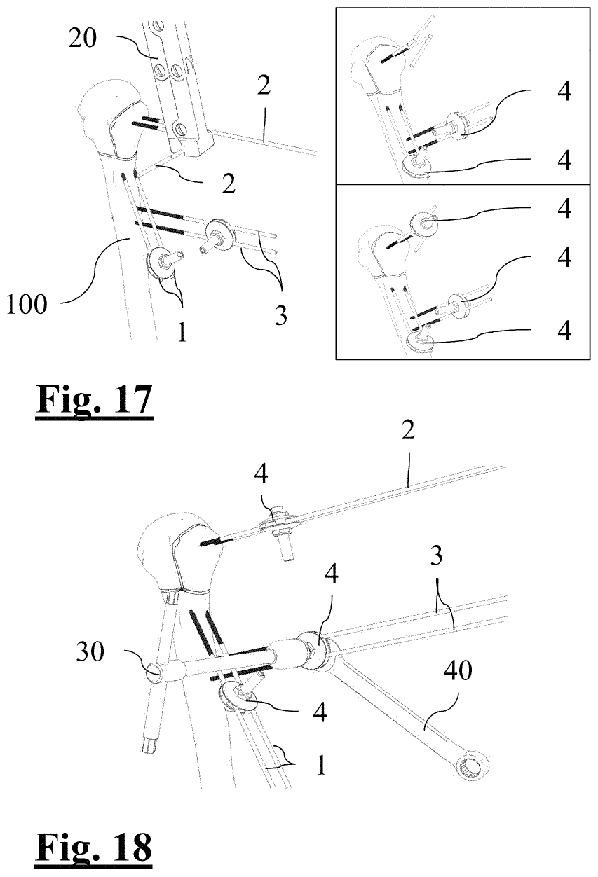

In order to do so, a first sub-step shown in FIGS. 17, 18 is that of stabilizing each wire pair (oblique 1, proximal transverse 2, distal transverse 3) by means of a respective wire locking clamp 4.

In doing so, the wire pairs are first sandwiched between the coupled disks of their respective wire locking clamp 4. If necessary, the wires are bent to approximately 90° with a wire bender 20. Then, as shown in FIG. 18, the wire locking clamps 9 are tightened by holding the clamp with an open-end wrench 30 and by turning the upper disk with a universal T-wrench 40.

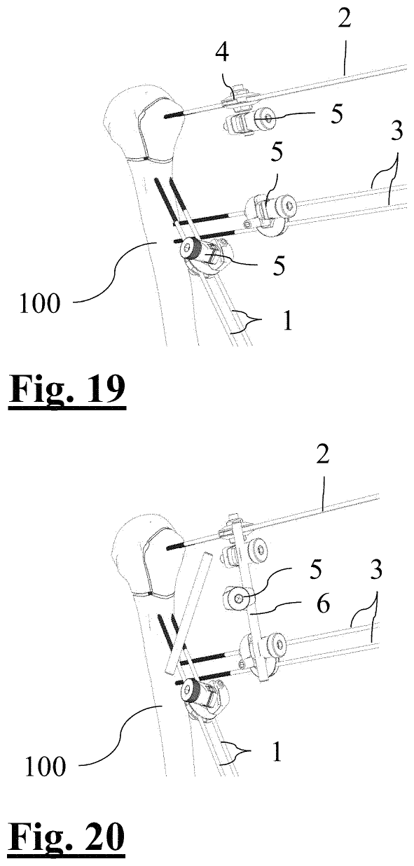

A second sub-step, illustrated in FIG. 19, is that of connecting each of the wire locking clamps 4 to a respective rod locking clamp 5. This is done by inserting the elongated stem 42 in one of the seats 53 of the rod locking clamp 5.

A third sub-step, illustrated in FIG. 20, is that of connecting the rod locking clamps 5 holding the proximal transverse wires 2 and the distal transverse wires 3 with a main rod 6. Also, an additional rod locking clamp 5 is attached to the main rod 6 in an intermediate position.

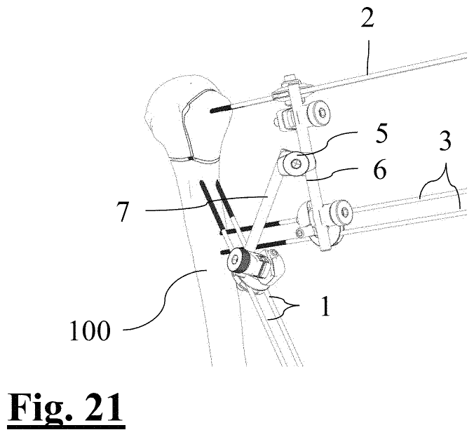

A fourth sub-step, illustrated in FIG. 21, is that of connecting the additional rod locking clamp 5 and the rod locking clamp holding the oblique wires 1 with an auxiliary rod 7.

Each of the rod locking clamp 5 must be pre-closed turning the tightening knob 52 fully and then tightened with a wrench for final closure.

After testing the stability of the fixation under image intensification, each wire 1, 2, 3 should be cut close to its wire locking clamp 4 and covered with a wire cover 11.

Obviously, the afore-described findings may be subjected to numerous modifications and variants—by a man skilled in the art with the aim of meeting the possible and specific requirements—all falling within the scope of protection of the invention as defined by the following claims.

1. Method for percutaneous fixation of a humeral head fracture, said method comprising the steps of:
inserting two oblique wires through the cortical bone of the humerus, said first oblique wires entering the bone at a site distal from the surgical neck and targeting the humeral head;
inserting two proximal transverse wires through the cortical bone of the humerus, said proximal transverse wires entering the bone at a site proximal to the surgical neck and targeting the humeral head;
inserting two distal transverse wires through the cortical bone of the humerus, said distal transverse wires entering the bone at a site distal to the surgical neck and targeting the humeral shaft;
connecting the oblique wires, the proximal transverse wires and the distal transverse wires to a common external fixator frame.

2. Method according to claim 1 wherein, in the step of inserting the oblique wires, each of the oblique wires is inserted until its tip is in the subchondral area of the humeral head.

3. Method according to claim 1 wherein, in the step of inserting the oblique wires, each wire is inserted from anterior to posterior.

4. Method according to claim 3, wherein, in the step of inserting the oblique wires, each wire is inserted with an inclination comprised between 15° and 25° with respect to the frontal plane.

5. Method according to claim 1, wherein: in the step of inserting the oblique wires, the two wires are inserted substantially parallel to each other; in the step of inserting the proximal transverse wires, the two wires are inserted substantially parallel to each other; in the step of inserting the distal transverse wires, the two wires are inserted substantially parallel to each other.

6. Method according to claim 1, comprising a preliminary step of performing a closed reduction of the fracture.

7. Method according to claim 6, wherein the distal transverse wires are inserted in the skin proximal to the oblique wires, whereas they enter the bone distally to the oblique wires.

8. Method according to claim 1, comprising a step of performing an open reduction of the fracture.

9. Method according to claim 8, wherein the steps of performing the open reduction of the fracture comprises making a deltopectoral incision, wherein the deltopectoral incision is kept open during at least one of the steps of inserting the oblique wires, inserting the distal transverse wires and inserting the proximal transverse wires.

10. Method according to claim 1, wherein the steps of connecting the oblique wires, the proximal transverse wires and the distal transverse wires to a common external fixator frame comprises a sub-step of connecting the proximal transverse wires and the distal transverse wires at the two opposite ends of a main rod.

11. Method according to claim 10, wherein the steps of connecting the oblique wires, the proximal transverse wires and the distal transverse wires to a common external fixator frame comprises a sub-step of connecting the oblique transverse wires to the main rod by means of an auxiliary rod, the auxiliary rod departing anteriorly with respect to the main rod.

12. Method according to claim 1, wherein the steps of connecting the oblique wires, the proximal transverse wires and the distal transverse wires to a common external fixator frame comprises a sub-step of connecting each pair of oblique, proximal transverse or distal transverse wires to a corresponding wire locking clamp.

13. Method according to claim 12, wherein the steps of connecting the oblique wires, the proximal transverse wires and the distal transverse wires to a common external fixator frame comprises a further step of connecting each wire locking clamp to a main or auxiliary rod by means of a rod locking clamp.

14. Method according to claim 1, wherein each of the oblique wires, the proximal transverse wires and the distal transverse wire is a bicortical wire.

15. Method according to claim 1, wherein each of the oblique wires, the proximal transverse wires and the distal transverse wire is partially threaded.

16. Method for percutaneous fixation of a humeral head fracture, said method comprising the steps of:
inserting two oblique wires through the cortical bone of the humerus shaft, said oblique wires being substantially oblique with respect to the humeral axis and crossing the surgical neck toward the humeral head;
inserting two proximal transverse wires through the cortical bone of the humerus, said proximal transverse wires being substantially perpendicular with respect to the humeral axis and being inserted into the humeral head;
inserting two distal transverse wires through the cortical bone of the humerus, said distal transverse wires being substantially perpendicular with respect to the humeral axis and being inserted into the humeral shaft;
connecting the oblique wires, the proximal transverse wires and the distal transverse wires to a common external fixator frame.

17. External fixation device comprising: two oblique wires arranged to be inserted into the humeral shaft, in a direction substantially oblique with respect to the humeral axis and crossing the surgical neck toward the humeral head; two proximal transverse wires arranged to be inserted into the humeral head in a direction substantially perpendicular with respect to the humeral axis; two distal transverse wires arranged to be inserted into the humeral shaft in a direction substantially perpendicular with respect to the humeral axis; and an external fixator frame stably connecting the oblique wires, the proximal transverse wires and the distal transverse wires.

18. External fixation device according to claim 17, wherein the external fixator frame comprises wire locking clamps for holding together each of the pair of oblique, proximal transverse and distal transverse wires.

19. External fixation device according to claim 18, wherein the external fixator frame further comprises a main and an auxiliary rod and rod locking clamps to connect the main and auxiliary rods together and each of the wire locking clamps to one of the main or auxiliary rods.

20. External fixation device according to claim 19, wherein the main rod is connected to the wire locking clamps relative to the proximal transverse and distal transverse wires, while the auxiliary rod is connected to the wire locking clamp relative to the oblique wires.
